(19) United States
(12) Patent Application Publication
(10) Pub. No.: US 2025/0325496 A1
(43) Pub. Date: Oct. 23, 2025

(54) SODIUM BICARBONATE IN SITU CONVERSION DRIVEN TRANSDERMAL DELIVERY OF AMINE DRUG

(71) Applicant: Corium, LLC, Cambridge, MA (US)

(72) Inventors: Eun Soo Lee, Redwood City, CA (US); Parminder Singh, Union City, CA (US); Appala Sagi, Mountain View, CA (US); Amit K. Jain, Milpitas, CA (US)

(21) Appl. No.: 18/922,900

(22) Filed: Oct. 22, 2024

Related U.S. Application Data
(63) Continuation of application No. 17/065,302, filed on Oct. 7, 2020, now abandoned.
Application No. 17/065,302 is a continuation of application No. 15/660,924, filed on Jul. 26, 2017, now abandoned.
(60) Provisional application No. 62/367,542, filed on Jul. 27, 2016.
(60) Provisional application No. 62/367,502, filed on Jul. 27, 2016.
(60) Provisional application No. 62/423,133, filed on Nov. 16, 2016.
(60) Provisional application No. 62/444,763, filed on Jan. 10, 2017.
(60) Provisional application No. 62/444,745, filed on Jan. 10, 2017.
(60) Provisional application No. 62/457,794, filed on Feb. 10, 2017.
(60) Provisional application No. 62/504,408, filed on May 10, 2017.
(60) Provisional application No. 62/504,391, filed on May 10, 2017.

(51) Int. Cl. A61K 9/70; A61K 31/00; A61K 31/045; A61K 31/13; A61K 31/137; A61K 31/18; A61K 31/27; A61K 31/445; A61K 47/02; A61K 47/10; A61K 47/12; A61K 47/32; C08K 5/00

(52) U.S. Cl. CPC A61K 9/7038; A61K 9/7023; A61K 9/7053; A61K 9/7061; A61K 9/7084; A61K 9/7092; A61K 31/00; A61K 31/13; A61K 31/137; A61K 31/18; A61K 31/27; A61K 31/445; A61K 47/02; A61K 47/10; A61K 47/12; A61K 47/32; C08K 5/0016; A61K 31/045

(57) ABSTRACT

Compositions, devices, and methods for transdermal administration of active agents provided in their salt form instead of neutral form are provided.

### CROSS-REFERENCE TO RELATED APPLICATIONS

This application is a continuation application of U.S. application Ser. No. 17/065,302, filed Oct. 7, 2020, which is a continuation application of U.S. application Ser. No. 15/660,924, filed Jul. 26, 2017, pending, which claims the benefit of U.S. Provisional Application No. 62/504,408, filed May 10, 2017; U.S. Provisional Application No. 62/504,391, filed May 10, 2017; U.S. Provisional Application No. 62/457,794, filed Feb. 10, 2017; U.S. Provisional Application No. 62/444,763, filed Jan. 10, 2017; U.S. Provisional Application No. 62/444,745, filed Jan. 10, 2017; U.S. Provisional Application No. 62/423,133, filed Nov. 16, 2016; U.S. Provisional Application No. 62/367,542, filed Jul. 27, 2016; and U.S. Provisional Application No. 62/367,502, filed Jul. 27, 2016, each herein incorporated by reference in its entirety.

### TECHNICAL FIELD

The subject matter described herein relates to compositions, devices, and methods for transdermal administration of amine active agents provided in their salt form instead of neutral form.

### BACKGROUND

Amine drugs exist in two forms, a free base and a salt. The salt form is the conjugated acid salt (i.e., protonated form) of an amine drug, and the free base is the conjugated base (i.e., deprotonated form) of the amine drug. In general, the salt form is more stable, water soluble, and bioavailable than the free base form. As such, most oral formulations of amine drugs include the salt form of the amine drug. In contrast, transdermal formulations typically use a free base form because the free base is much more skin permeable than the salt form.

There are a several significant drawbacks, however, in using the free base form in a transdermal formulation for many drugs. For example, it is often difficult to solubilize a sufficient amount of free base in a drug-in-polymer matrix because the free base often has low solubility in the polymer matrix and tends to recrystallize into solid crystals during processing or during storage prior to use. Further, certain liquid free base drugs are volatile, and a significant amount of the drug can be lost during processing due to evaporation. Additionally, drug flux is often difficult to control and deliver for multiple days at a constant rate when a drug is very permeable through the skin. Finally, drugs are often more unstable in the free base form than in the salt form.

Researchers have attempted to formulate a salt form of a drug with a basic inorganic salt, so that it converts into a free base in situ in a drug-in-adhesive matrix in a transdermal formulation. In those prior attempts, the basic inorganic salts had a higher pKa value than the conjugated acid salt form of the amine drug, and as such, the converted free base was not soluble in the matrix and recrystallized into solid crystals, leading to decreased skin permeation.

There is a need in the art for improved compositions, devices, patches, systems, and methods for transdermal delivery of amine drugs that address these shortcomings.

### BRIEF SUMMARY

The following aspects and embodiments thereof described and illustrated below are meant to be exemplary and illustrative, not limiting in scope.

In one aspect, a composition for transdermal delivery is provided. The composition comprises a drug reservoir (also referred to as an adhesive matrix) comprising an amine salt form of an active agent and an amphoteric inorganic base compound, wherein the pKa of the amphoteric inorganic base compound is lower than that of the amine salt form of the active agent.

In another aspect, a composition for transdermal delivery is provided. The composition comprises an adhesive, an amine salt form of an active agent and an amphoteric inorganic base compound, wherein the pKa of the amphoteric inorganic base compound is lower than that of the amine salt form of the active agent.

In one embodiment, the amphoteric inorganic base compound is sodium bicarbonate.

In one embodiment, the active agent is donepezil, memantine, fentanyl, oxybutynin, rotigotine, ropinirole, rivastigmine, tamsulosin, methylphenidate, or buprenorphine.

In another embodiment, the drug reservoir comprises between about 5-35% w/w of the active agent.

In yet another embodiment, the composition comprises between about 0.5-35% w/w of sodium bicarbonate.

In still another embodiment, the composition further comprises a salt form solubilizer selected from the group consisting of water, alcohols, glycerol, propylene glycol, ethylene glycol, dimethyl sulfoxide, and N-methylpyrrolidone.

In one embodiment, the drug reservoir or composition comprises up to 15% w/w of the salt form solubilizer.

In still another embodiment, the composition comprises a neutral form solubilizer selected from the group consisting of a fatty acid ester, a dicarboxylic acid ester, a glycerol ester, a lactate, a fatty alcohol, sorbitan monolaurate, sorbitan monooleate, lauryl lactate, propylene glycol monolaurate, dimethyl succinate, lauryl alcohol, and oleyl alcohol.

In one embodiment, the composition comprises up to 20% w/w of the neutral form solubilizer.

In one embodiment, the composition further comprises a plasticizer selected from the group consisting of a dicarboxylic acid ester, an adipate, a sebacate, a maleate, a tricarboxylic ester, triethyl citrate, tributyl citrate, a glycerol ester, and triacetin.

In another embodiment, the composition comprises up to 20% w/w of the plasticizer.

In another embodiment, the composition further comprises a matrix modifying additive selected from the group consisting of crospovidone and colloidal silicone dioxide.

In one embodiment, the composition comprises up to 25% w/w of the matrix modifying additive.

In another embodiment, the composition comprises an adhesive agent selected from the group consisting of an acrylate, polyisobutylene, silicone adhesive, and styrene block copolymer based adhesive.

In one embodiment, the adhesive comprises up to 65% w/w of the composition.

In another aspect, a transdermal patch is provided, where the patch comprises a composition as described herein as a first drug reservoir and a backing layer.

In one embodiment, the backing layer is an occlusive polymer film.

In other embodiments, the transdermal patch comprises a contact adhesive layer comprised of an adhesive selected from the group consisting of an acrylate, polyisobutylene, silicone adhesive, and styrene block copolymer based adhesive.

In still other embodiments, the transdermal patch comprises a nonwoven tie layer between the drug reservoir and the contact adhesive layer.

In still other embodiments, the transdermal patch comprises a rate-controlling membrane between the drug reservoir and the contact adhesive layer.

In still other embodiments, the transdermal patch comprises a second drug reservoir comprised of a composition or adhesive matrix as described herein.

In still other embodiments, the first drug reservoir and second drug reservoir are separated by a nonwoven tie layer.

In other embodiments, the first drug reservoir and second drug reservoir are separated by a rate-controlling membrane.

A method of transdermally administering an active agent to a patient in need thereof, comprising: providing a composition or a transdermal patch as described herein to a patient in need thereof.

In other aspects, a method for treating Alzheimer's disease, Parkinson's disease, restless leg syndrome, attention deficit hyperactivity disorder, narcolepsy, depression, anxiety disorder, obsessive compulsive disorder, benign prostatic hyperplasia, acute urinary retention, opioid dependence, moderate acute pain in non-opioid-tolerant individuals, or moderate chronic pain are provided. The method comprises providing a composition or a transdermal patch as described herein to a patient in need thereof.

The method may further include administering or instructing to administer to the skin of the patient the composition or transdermal patch.

In some embodiments, administering achieves a therapeutically effective blood concentration of the active agent. In some embodiments, the therapeutically effective blood concentration of the active agent is achieved for a period of at least about 3 days, 5 days or 7 days.

### BRIEF DESCRIPTION OF THE FIGURES

FIG. 1 is a chemical reaction schematic depicting how sodium bicarbonate drives in situ conversion and transdermal delivery of an amine drug.

FIGS. 2-5 are illustrations of exemplary embodiments of transdermal patch configurations.

FIG. 6 is a graph of average skin flux for donepezil transdermal delivery devices, in μg/cm²·hr, in vitro as a function of time, in hours, in an in vitro skin permeation test for devices having a formulation according to Example 1.

FIG. 7 is a graph of average skin flux for memantine transdermal delivery devices, in μg/cm²·hr, in vitro as a function of time, in hours, in an in vitro skin permeation test for devices having formulations according to Example 2 (squares), Comparative Example 3 (diamonds), Comparative Example 4 (circles), and Comparative Example 5 (triangles).

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Compositions, devices, and methods described herein are not limited to the specific polymers, excipients, cross-linking agents, additives, manufacturing processes, or adhesive products described herein. It will be understood that the particular terminology used herein is for the purpose of describing particular embodiments and is not intended to be limiting.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to a “solvent” includes a single solvent as well as two or more of the same or different solvents, and the like.

The use of terms of order or importance, including “first” and “second,” is to distinguish and identify individual elements and does not denote or imply a particular order or importance unless clearly indicated by context.

The term “active agent” as used herein refers to a chemical material or compound suitable for topical or transdermal administration and that induces a desired effect. The terms include agents that are therapeutically effective, prophylactically effective, and cosmetically effective agents. The terms “active agent,” “drug,” and “therapeutic agent” are used interchangeably herein.

An “adhesive matrix” as described herein includes matrices made in one piece, for example, matrices made via solvent casting or extrusion as well as matrices formed in two or more portions that are then pressed or joined together.

The term “skin” as used herein refers to skin or mucosal tissue, including the interior surface of body cavities that have a mucosal lining. The term “skin” should be interpreted as including “mucosal tissue” and vice versa.

The term “therapeutically effective amount” as used herein refers to the amount of an active agent that is nontoxic but sufficient to provide the desired therapeutic effect. The amount that is “effective” will vary from subject to subject, depending on the age and general condition of the individual, the particular active agent or agents, and the like as known to those skilled in the art.

The terms “transdermal” or “transdermal delivery” as used herein refer to administration of an active agent to a body surface of an individual so that the agent passes through the body surface (e.g., through the skin) and into the individual's blood stream. The term “transdermal” is intended to include transmucosal administration, i.e., administration of a drug to the mucosal (e.g., sublingual, buccal, vaginal, rectal, etc.) surface of an individual so that the agent passes through the mucosal tissue and into the individual's blood stream.

### II. Compositions/Devices

Compositions and/or devices are provided for transdermal administration of active agents. Compositions may be used in devices, patches, and/or systems for transdermal delivery of one or more active agents. Compositions described herein are contemplated for use in transdermal delivery systems, devices, patches, and/or methods as described herein.

In general, compositions described herein provide an active agent as an amine salt, and an amphoteric inorganic base compound whose dissociation constant (pKa) is lower than the pKa of the conjugated acid salt of the amine drug, such as sodium bicarbonate. Carbonic acid has two pKa values, 6.4 and 10.3. In the presence of stronger base, it reacts as an acid by donating a proton and forming a carbonate ion and water (see Equation 1), with a pKa of 10.3.

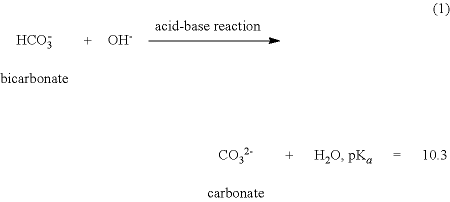

But in the presence of a weak acid, sodium bicarbonate acts as a base and forms carbonic acid, which is unstable and likely to dissociate into carbon dioxide and water (see Equation 2). The dissociation constant of carbonic acid is 6.4.

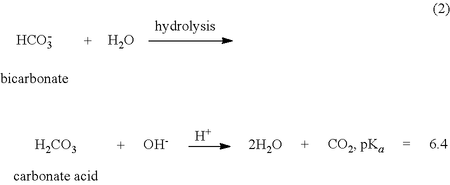

Most conjugated acid amine drugs (depicted herein as “AMNH⁺”) are weak acids, and thus sodium bicarbonate acts as a base in their presence, generating reaction products of carbonic acid (which rapidly dissociates into water and CO₂) and the free base of the amine drug (depicted herein as “AMN”). Most conjugated acid amine drugs, however, have greater pKa values than does sodium bicarbonate (i.e., they are more basic than sodium bicarbonate), which means that the reaction equilibrium favors the conjugated acid amine drug and bicarbonate ion reactants, and only a small amount of carbonic acid and amine drug free base are formed. Equation 3 depicts these reactions:

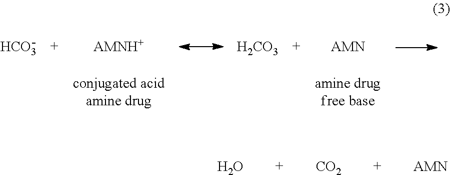

The equilibrium constant between carbonic acid and bicarbonate, K_{a(carbonic acid)}, is:

$${K}_{a ( carbonic acid )} = \frac{[ {HCO}_{3}^{-} ] [ {H}^{+} ]}{[ {H}_{2} {CO}_{3} ]} \qquad ( 4 )$$

The equilibrium constant between the conjugated acid amine AMNH⁺ and the conjugated free amine AMN is:

$${K}_{a ( acid amine )} = \frac{[ AMN ] [ {H}^{+} ]}{[ AMN {H}^{+} ]} \qquad ( 5 )$$

The greater the ratio of

$$\frac{{K}_{a ( acid amine )}}{{K}_{a ( carbonic acid )}}$$

is, the more AMN is produced per Equation 3. This ratio is calculated from Equations 4 and 5, and is shown in FIG. 5:

$$\frac{{K}_{a ( acid amine )}}{{K}_{a ( carbonic acid )}} = \frac{[ {H}_{2} {CO}_{3} ] [ AMN ]}{[ {HCO}_{3}^{-} ] [ AMN {H}^{+} ]} = \frac{[ {H}_{2} O ] [ {CO}_{2} ] [ AMN ]}{[ {HCO}_{3}^{-} ] [ AMN {H}^{+} ]} \qquad ( 6 )$$

Table 1 presents K_{a(acid amine)}/K_{a(carbonic acid)} ratios for several exemplary amine drugs, assuming an aqueous medium and that pK_{a(carbonic acid)}=6.4.

TABLE 1
Equilibrium Ratios of Exemplary Conjugated Acid Salt Amines and
Conjugated Free Amine in the Presence of Sodium Bicarbonate.

| | Equilibrium Constant | Ratio of |
|---|---|---|
| Conjugated Acid Amine | of Acid Amine (pK_{a(acid amine)}) | $\frac{{K}_{a ( acid amine )}}{{K}_{a ( carbonic acid )}}$ |
| Donepezil HCl | 8.6 | 0.006310 |
| Memantine HCl | 10.27 | 0.000135 |
| Rotigotine HCl | 7.90 | 0.031623 |
| Ropinirole HCl | 9.68 | 0.000525 |
| Rivastigmine HCl | 8.85 | 0.003548 |
| Tamsulosin HCl | 8.37 | 0.010715 |
| Methyl phenidate HCl | 8.77 | 0.004266 |
| Buprenorphine HCl | 8.31 | 0.012303 |

The ratios presented in Table 1 are very small for all exemplary amine drugs shown. As such, a reaction according to Equation 3 involving the exemplary amine drugs listed in Table 1 will favor the reactants, and only a very small amount of amine drug free base product will be formed. Because of this, conjugated free base amine is not precipitated out as crystals, but instead, remains solubilized in the equilibrium system and ultimately is removed by diffusion and permeation through the skin. As the conjugated free base amine is removed by permeation through the skin, the reaction is continuously driven forward to generate more amine drug free base to maintain equilibrium.

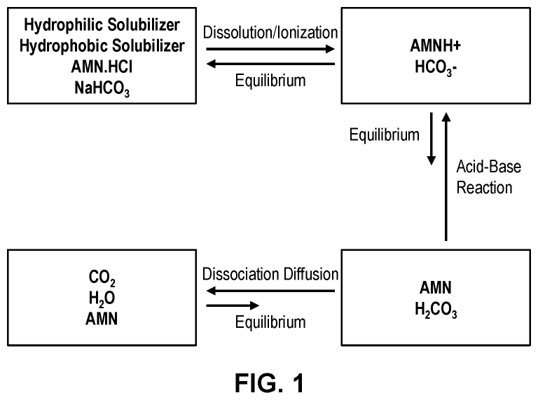

FIG. 1 provides a schematic representation of the chemical reactions driven by certain compositions described herein. The box in the top left represents ingredients in certain compositions, i.e., an amine salt of an active agent (“AMN·HCl”), sodium bicarbonate (NaHCO₃), a salt form solubilizer (“hydrophilic solubilizer”), and a neutral form solubilizer (“hydrophobic solubilizer”). In the first step of the reaction, AMN·HCl and sodium bicarbonate are ionized to form a conjugated acid amine ion (“AMNH⁺”), its counterion (e.g., a chloride or other ion with a single negative charge), a bicarbonate ion (HCO₃⁻), and its counterion (Na⁺) (see the box at the top right of FIG. 1). The salt form solubilizer helps to solubilize and stabilize these ionized reaction products.

The arrows connecting the boxes at the top right and lower right of FIG. 1 depict the equilibrium between the ionized conjugated acid amine ion and bicarbonate ion (top right) and the conjugated free base amine (“AMN”) and carbonic acid (H₂CO₃) (bottom right). The neutral form solubilizer helps to stabilize the conjugated free base amine. The equilibrium of this step favors the reactants, AMNH⁺ and bicarbonate (top right).

FIG. 1 depicts the last phase in the reaction scheme, in which AMN and carbonic acid are cleared from the reaction system. AMN is cleared by diffusion and permeation into the skin. Carbonic acid is cleared by its dissociation into water and carbon dioxide gas. The equilibrium in this last step of the reaction scheme favors AMN, water, and carbon dioxide.

### A. Compositions for Transdermal Delivery of Active Agents

In some aspects, provided are compositions comprising an adhesive or polymer, an amine salt form of at least one active agent and at least one amphoteric inorganic base compound whose pKa is lower than the pKa of the amine salt drug, such as sodium bicarbonate. In general, the amine salt form of a provided active agent and the amphoteric inorganic base will ionize, and the ionized, positively charged active agent will react with the ionized, negatively charged inorganic base compound to generate a neutral form of the active agent that is more skin permeable than the salt form.

In some aspects, compositions comprising an adhesive, an amine salt form of at least one active agent and sodium bicarbonate are provided. In general, the amine salt form of a provided active agent and the amphoteric inorganic base will ionize, and the ionized, positively charged active agent will react with the negatively charged bicarbonate ion to generate a neutral form of the active agent that is more skin permeable than the salt form.

In some embodiments, an adhesive composition may include one or more additional ingredients that cause the neutral form of the active agent to be generated at a specified and/or desired rate. In some embodiments, such compositions can provide a relatively constant activity of an active agent. In some embodiments, compositions may further include one or more of the following: at least one plasticizer, at least one salt form solubilizer, at least one neutral form solubilizer, adhesive matrix modifying additive, and adhesive polymer.

It will be appreciated that all w/w % or wt % described herein may refer to wet or dry weight of the composition.

In some embodiments, compositions comprised of micronized particles of a salt form of one or more amine-containing active agents dispersed in an adhesive matrix are provided. In some embodiments, an adhesive matrix comprises about 1-70 wt %, about 1-50 wt %, about 1-35 wt %, about 1-25 wt %, about 2-70 wt %, about 2-50 wt %, about 2-35 wt %, about 5-70 wt %, about 5-50 wt %, about 5-35 wt %, about 5-30 wt %, about 5-25 wt %, about 5-20 wt %, about 5-15 wt %, about 5-10 wt %, about 10-35 wt %, about 10-30 wt %, about 10-25 wt %, about 10-20 wt %, about 10-15 wt %, about 20-35 wt %, about 20-30 wt %, about 20-25 wt %, about 25-35 wt %, about 25-30 wt %, or about 30-35 wt % of a salt form of at least one active agent.

In some embodiments, the adhesive matrix composition further comprises a solubilizer that has a limited solubility for the salt form of the active agent (the “salt form solubilizer”). In some embodiments, the micronized particles of a salt form of an active agent will ionize in the salt form solubilizer. In some embodiments, the micronized salt form particles will be maintained in equilibrium with the dissolved, ionized salt form. In some embodiments, the salt form solubilizer has only a limited degree of solubility for the micronized salt particles, such that the equilibrium favors the micronized salt particles over the dissolved, ionized salt form.

In some embodiments, a salt form solubilizer has a solubility for the salt of at least about 0.1% w/w, at least about 0.2% w/w, at least about 0.3% w/w, at least about 0.4% w/w, at least about 0.5% w/w, or at least about 1.0% w/w. In some embodiments, the salt form solubilizer has a solubility for the salt of less than 30% w/w or less than about 25% w/w or 20% w/w.

In some embodiments, a salt form solubilizer is a protic solvent (e.g., a solvent that has a hydrogen atom bound to an oxygen (e.g., as in a hydroxyl group) or a nitrogen (e.g., as in an amine group), and/or any solvent that contains labile protons). Exemplary salt form solubilizers include, but are not limited to, water, alcohols (e.g., ethanol, methanol, etc.), glycerol, propylene glycol, ethylene glycol, dimethyl sulfoxide, N-methylpyrrolidone, and/or combinations thereof.

In some embodiments, the adhesive matrix composition comprises about 0-50 wt %, about 0-20 wt %, about 0-10 wt %, about 0-5 wt %, about 1-50 wt %, about 1-20 wt %, about 2-50 wt %, about 2-20 wt %, about 5-50 wt %, about 5-20 wt %, about 5-15 wt %, about 5-10 wt %, or about 10-15 wt % of at least one salt form solubilizer.

In some embodiments, the compositions further comprise one or more solubilizers for the neutral form of the active agent (a “neutral form solubilizer”). In some embodiments, the neutral form solubilizer helps ensure that a neutral active agent, once formed, can persist long enough to diffuse into the skin.

In some embodiments, a neutral form solubilizer has a solubility for the neutral form of the active agent of at least about 0.1% w/w. In some embodiments, the neutral form solubilizer has a solubility for the neutral form of the active agent of less than 30% w/w.

In some embodiments, exemplary neutral form solubilizers generally include, but are not limited to, fatty acid esters, lactate esters, dicarboxylic esters, citrate esters, glycerol esters, fatty alcohols, and/or combinations thereof. In some embodiments, exemplary neutral form solubilizers include, but are not limited to, sorbitan monooleate, sorbitan monolaurate (SPAN® 20), propylene glycol monolaurate, lauryl lactate, dimethyl succinate, triethyl citrate, triacetin, isopropyl myristate, isopropyl palmitate, octyl dodecanol, lauryl alcohols, oleyl alcohols, and/or combinations thereof.

In some embodiments, an adhesive matrix comprises 0-40 wt %, about 0-30 wt %, about 0-20 wt %, about 0-15 wt %, about 0-10 wt %, about 0-5 wt %, about 1-40 wt %, about 1-30 wt %, about 1-20 wt %, about 2-40 wt %, about 2-30 wt %, about 2-20 wt %, about 5-20 wt %, about 1-15 wt %, about 2-15 wt %, about 5-15 wt %, about 5-10 wt %, or about 10-15 wt % of least one neutral form solubilizer.

In some embodiments, active agents include amine salt drugs.

In some embodiments, the active agent is an amine salt drug. In some embodiments, amine salt drugs have a solubility of at least about 0.1 mg/g, at least about 0.2 mg/g, at least about 0.3 mg/g, at least about 0.4 mg/g, at least about 0.5 mg/g, or at least about 1.0 mg/g in the adhesive matrix. In some embodiments, amine salt drugs have a solubility of less than about 100 mg/g in the adhesive matrix. Exemplary amine salt drugs include, but are not limited to, donepezil, memantine, rotigotine, ropinirole, rivastigmine, tamsulosin, methylphenidate, buprenorphine, fentanyl, fingolimod, and oxybutynin.

In some embodiments, a composition for transdermal delivery of an active agent further comprises one or more plasticizers. In some embodiments, salt form solubilizers and/or neutral form solubilizers already present in the composition may also serve as plasticizers. In such embodiments, it may not be necessary to include an additional plasticizer. In some embodiments, a plasticizer that does not also serve as a salt form and/or neutral form solubilizer is included in a composition. Exemplary plasticizers include, but are not limited to, dicarboxylic acid esters (e.g., adipates, sebacates, maleates, etc.), tricarboxylic esters (e.g., triethyl citrate, tributyl citrate, etc.), esters of glycerol (e.g., triacetin, etc.), and/or combinations thereof.

In some embodiments, an adhesive matrix comprises about 0-20 wt %, about 0-15 wt %, about 0-10 wt %, about 0-5 wt %, about 5-20 wt %, about 5-15 wt %, about 5-10 wt %, about 10-20 wt %, about 10-15 wt %, or about 15-20 wt % of at least one plasticizer.

In some embodiments, a composition for transdermal delivery of an active agent further comprises at least one adhesive modifying additive, also referred to as a matrix modifying additive. In some embodiments, matrix modifying additives modify cohesion and/or diffusivity of described active agent compositions. In some embodiments, the matrix modifying additive modifies the solubility of the active agent in the adhesive matrix. In some embodiments, matrix modifying additives can absorb moisture and/or water emanating from the skin under occlusion, which improves adhesion to the skin. In some embodiments, matrix modifying additives facilitate homogenization of the adhesive matrix. Exemplary matrix modifying additives include, but are not limited to, crospovidone (KOLLIDON® CL-M, etc.), cross-linked polyvinylpyrrolidone (PVP), a soluble polyvinylpyrrolidone (PVP), fumed silica, colloidal silicone dioxide, a cellulose derivative (e.g. hydroxypropyl cellulose (HPC), hydroxyethylcellulose (HEC)), a polyacrylamide, a polyacrylic acid, a polyacrylic acid salt, a clay (e.g., kaolin, bentonite, etc.), and/or combinations thereof. An exemplary commercial fumed silica product is AEROSIL® 200P, an amorphous, anhydrous colloidal silicon dioxide (Evonik Industries). Another exemplary fumed silica product is Cab-O-Sil® (Cabot Corporation, Boston, Mass.).

In some embodiments, an adhesive matrix comprises about 0-25 wt %, about 0-20 wt %, about 0-15 wt %, about 0-10 wt %, about 0-5 wt %, about 5-25 wt %, about 5-20 wt %, about 5-15 wt %, about 5-10 wt %, about 10-25 wt %, about 10-20 wt %, about 10-15 wt %, about 15-25 wt %, about 15-20 wt %, or about 20-25 wt % of at least one adhesive matrix modifying additive.

In some embodiments, a composition for transdermal delivery of an active agent further comprises at least one adhesive or adhesive polymer. Exemplary adhesives include, but are not limited to, acrylates, polyisobutylene, silicone adhesives, styrene block copolymer based adhesives, and/or combinations thereof.

In some embodiments, an adhesive matrix comprises about 0-65 wt %, about 0-60 wt %, about 0-55 wt %, about 0-50 wt %, about 0-45 wt %, about 0-40 wt %, about 0-35 wt %, about 0-30 wt %, about 0-25 wt %, about 0-20 wt %, about 0-15 wt %, about 0-10 wt %, about 0-5 wt %, 5-65 wt %, about 5-60 wt %, about 5-55 wt %, about 5-50 wt %, about 5-45 wt %, about 5-40 wt %, about 5-35 wt %, about 5-30 wt %, about 5-25 wt %, about 5-20 wt %, about 5-15 wt %, about 5-10 wt %, 10-65 wt %, about 10-60 wt %, about 10-55 wt %, about 10-50 wt %, about 10-45 wt %, about 10-40 wt %, about 10-35 wt %, about 10-30 wt %, about 10-25 wt %, about 10-20 wt %, about 10-15 wt %, 15-65 wt %, about 15-60 wt %, about 15-55 wt %, about 15-50 wt %, about 15-45 wt %, about 15-40 wt %, about 15-35 wt %, about 15-30 wt %, about 15-25 wt %, about 15-20 wt %, 20-65 wt %, about 20-60 wt %, about 20-55 wt %, about 20-50 wt %, about 20-45 wt %, about 20-40 wt %, about 20-35 wt %, about 20-30 wt %, about 20-25 wt %, 25-65 wt %, about 25-60 wt %, about 25-55 wt %, about 25-50 wt %, about 25-45 wt %, about 25-40 wt %, about 25-35 wt %, about 25-30 wt %, 30-65 wt %, about 30-60 wt %, about 30-55 wt %, about 30-50 wt %, about 30-45 wt %, about 30-40 wt %, about 30-35 wt %, 35-65 wt %, about 35-60 wt %, about 35-55 wt %, about 35-50 wt %, about 35-45 wt %, about 35-40 wt %, 40-65 wt %, about 40-60 wt %, about 40-55 wt %, about 40-50 wt %, about 40-45 wt %, 45-65 wt %, about 45-60 wt %, about 45-55 wt %, about 45-50 wt %, 50-65 wt %, about 50-60 wt %, about 50-55 wt %, 55-65 wt %, about 55-60 wt %, or about 60-65 wt % of at least one adhesive polymer.

The composition may also include other conventional additives such as adhesive agents, antioxidants, crosslinking agents, curing agents, pH regulators, pigments, dyes, refractive particles, conductive species, antimicrobial agents, opacifiers, gelling agents, viscosity modifiers, thickening agents, stabilizing agents, permeation enhancers, and the like as known in the art. In those embodiments wherein adhesion needs to be reduced or eliminated, conventional detackifying agents may also be used. In some embodiments, agents such as antimicrobial agents are included to prevent spoilage upon storage, e.g., to inhibit growth of microbes such as yeasts and molds. Suitable antimicrobial agents are typically selected from the group consisting of the methyl and propyl esters of p-hydroxybenzoic acid (e.g., methyl and propyl paraben), sodium benzoate, sorbic acid, imidurea, and/or combinations thereof. These additives, and amounts thereof, are selected in such a way that they do not significantly interfere with the desired chemical and physical properties of the adhesive and/or active agent.

Compositions may also contain irritation-mitigating additives to minimize or eliminate the possibility of skin irritation and/or skin damage resulting from the active agent, the proton accepting entity, salt form solubilizer, neutral form solubilizer, plasticizer, matrix modifying additive, adhesive and/or other components of the composition. Suitable irritation-mitigating additives include, for example: corticosteroids; α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine; and/or combinations thereof.

Methods for preparing or manufacturing active agent compositions are also provided. Exemplary methods are set forth in the Examples section. Methods for preparing active agent compositions generally involve mixing an active agent with sodium bicarbonate, optionally along with a salt form solubilizer, a neutral form solubilizer, a matrix modifying additive, a plasticizer, an adhesive polymer, and/or combinations thereof.

### B. Transdermal Devices

In certain aspects, compositions are provided in transdermal devices (e.g., patches). In general, transdermal patches comprise a backing layer, at least one drug reservoir, and a contact adhesive layer. In some embodiments, transdermal patches further comprise one or more release liners, tie layers, rate-controlling membranes, and/or various combinations of the foregoing.

In some embodiments, transdermal patches comprise one or more of the following components: backing layer, drug reservoir, contact adhesive layer, release liner, tie layer, rate-controlling membrane, and/or various combinations of the foregoing.

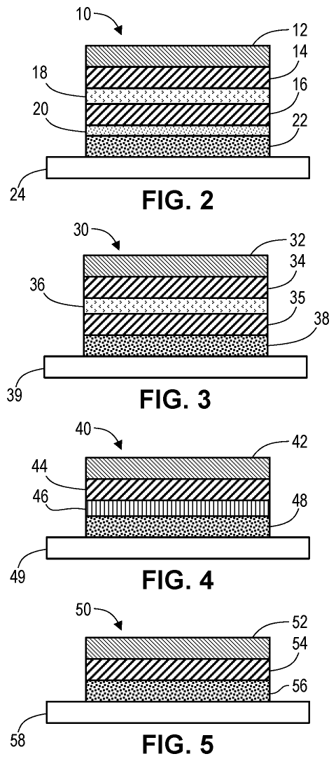

Exemplary transdermal patches are shown in FIGS. 2-5. FIG. 2 shows an exemplary transdermal patch 10 comprising a backing layer 12, multiple drug reservoirs 14, 16 separated by a nonwoven tie layer 18, a rate-controlling membrane 20, a contact adhesive layer 22, and a release liner 24. This particular example presents multiple drug reservoirs separated by a tie layer, but in some embodiments, multiple adhesive drug reservoir layers may be in direct contact with each other without a tie layer. In such embodiments wherein the transdermal patch comprises multiple drug reservoirs, each drug reservoir may comprise the same or different active agents. In such embodiments wherein the transdermal patch comprises multiple drug reservoirs, each drug reservoir may comprise different concentrations of the same active agent. This particular example presents a rate-controlling membrane between the drug reservoirs and the release liners, but in some embodiments, the rate-controlling membrane is not present. FIG. 3 presents such an embodiment, in which the transdermal system 30 comprises a backing layer 32, multiple drug reservoirs 34, 35 separated by a nonwoven tie layer 36, a contact adhesive layer 38, and a release liner 39, but no rate-controlling membrane between the drug reservoirs and the contact adhesive.

FIG. 4 shows an exemplary transdermal patch 40 comprising a backing layer 42, a drug reservoir 44, a tie layer or rate-controlling membrane 46, a contact adhesive layer 48, and a release liner 49. In some embodiments, a transdermal patch includes a nonwoven tie layer between the drug reservoir and the contact adhesive layer. In some embodiments, a transdermal patch includes a rate-controlling membrane between the drug reservoir and the contact adhesive layer. In some embodiments, a transdermal patch includes both a nonwoven tie layer and a rate-controlling membrane between the drug reservoir and the contact adhesive layer. In some embodiments, a transdermal patch does not include either a nonwoven tie layer or a rate-controlling membrane between the drug reservoir and the contact adhesive layer. FIG. 5 presents such an embodiment, in which the patch 50 comprises a backing layer 52, a drug reservoir 54, a contact adhesive layer 56, and a release liner 58, but no tie layer or rate-controlling membrane between the drug reservoir and the contact adhesive layer.

In some embodiments, a backing layer provides a structural element for holding or supporting the adhesive layer. A backing layer may be formed of any suitable material as known in the art. In some embodiments, a backing layer is occlusive. In some embodiments, a backing layer is preferably impermeable or substantially impermeable to moisture. In one exemplary embodiment, the barrier layer has an MVTR (moisture vapor transmission rate) of less than about 50 g/m²-day. In some embodiments, a backing layer is preferably inert and/or does not absorb components of the adhesive layer, including the active agent. In some embodiments, a backing layer preferably prevents release of components of the adhesive layer through the backing layer. A backing layer may be flexible or nonflexible. A backing layer is preferably at least partially flexible such that the backing layer is able to conform at least partially to the shape of the skin where the patch is applied. In some embodiments, a backing layer is flexible such that the backing layer conforms to the shape of the skin where the patch is applied. In some embodiments, a backing layer is sufficiently flexible to maintain contact at the application site with movement, e.g., skin movement. Typically, the material used for a backing layer should permit the device to follow the contours of the skin or other application site and be worn comfortably on areas of skin such as at joints or other points of flexure, that are normally subjected to mechanical strain with little or no likelihood of the device disengaging from the skin due to differences in the flexibility or resiliency of the skin and the device.

In some embodiments, a backing layer is formed of one or more of a film, non-woven fabric, woven fabric, laminate, and combinations thereof. In some embodiments, the film is a polymer film comprised of one or more polymers. Suitable polymers are known in the art and include, but are not limited to, elastomers, polyesters, polyethylene, polypropylene, polyurethanes, polyether amides, and/or combinations thereof. In some embodiments, a backing layer is formed of one or more of polyethylene terephthalate, various nylons, polypropylene, metalized polyester films, polyvinylidene chloride, aluminum foil, and/or combinations thereof. In some embodiments, a backing layer is a fabric formed of one or more of polyesters such as polyethylene terephthalate, polyurethane, polyvinyl acetate, polyvinylidene chloride, polyethylene, and/or combinations thereof. In one particular, but non-limiting embodiment, the backing layer is formed of a polyester film laminate. Exemplary particular polyester film laminates include, but are not limited to, the polyethylene and/or polyester laminates such as those sold under the names Scotchpak™ #9723, Scotchpak™ #1012, and the like.

In some embodiments, the drug reservoir generally comprises a salt form of an active ingredient(s) (total 5-35% w/w), at least one salt form solubilizer (total 0-15% w/w), at least one neutral form solubilizer (total 0-15% w/w), at least one proton accepting entity a (total 0.5-30% w/w), matrix modifying additives (total 0-25% w/w), and optionally adhesive polymers (total 0-65% w/w). In some embodiments, the drug reservoir comprises any of the compositions for transdermal delivery described herein, e.g., in the Examples and in Section II.A.

In general, a tie layer comprises a nonwoven fabric, porous polyethylene film and/or a rate controlling polymer membrane.

In some embodiments, devices further include one or more fabric or tie layers within or between the adhesive layers. It will be appreciated that a tie layer may be included between one, some, or all of the adhesive matrix layers. In some embodiments, a tie layer is useful to increase bonding between layers of the device. Tie layers may increase bonding by providing chemical groups for the polymers to bind. In some embodiments, a tie layer is useful as a separation for adhesive matrix layers.

In some embodiments, a tie layer does not affect the rate of release of an active agent from the adhesive layers. In some embodiments, tie layers may comprise nonwoven films that include, but are not limited to, nylon, cotton, porous polyethylene, and the like, and/or combinations thereof.

In some embodiments, tie layers may comprise rate controlling polymer membranes. Exemplary rate controlling polymer membranes include, but are not limited to, microporous polymer films such as CELGARD® 2400 (microporous polypropylene), polyethylenes (e.g., microporous polyethylene), polyesters (e.g., REEMAY®), vinyl acetate polymers and copolymers, and the like, and/or combinations thereof. In general, a rate controlling polymer membrane allows for a rate-controlled release of the drug from the drug reservoir layer.

In some embodiments, the tie layer comprises a nonwoven fabric and does not comprise a rate controlling polymer membrane. In some embodiments, a tie layer comprises a rate controlling polymer membrane and does not comprise a nonwoven fabric. In some embodiments, a tie layer comprises both a nonwoven fabric and a rate controlling polymer membrane. To give but one example, a nonwoven fabric 18 and a rate controlling polymer membrane 20 may both be used when the tie layer is embedded within the drug reservoir (14, 16) to help improve drug reservoir cohesion (see, e.g., FIG. 2).

The device includes at least one adhesive layer. In embodiments, at least one of the adhesive layers is an adhesive matrix comprising one or more active agents as described below. The adhesive layer adheres to a drug reservoir, an adjacent adhesive layer, a tie layer, a release liner, and/or skin at the administration site. In some embodiments, an adhesive layer serves to release the active agent to the skin. In some embodiments, one or more of the drug reservoir adhesive and/or the contact layer adhesive are formed of an adhesive matrix. Exemplary adhesives include, but are not limited to, acrylates, polyisobutylene, a silicone adhesive, a styrene block copolymer based adhesive, or the like, and/or combinations thereof.

In some embodiments, the delivery system provides an in vitro skin flux of an active agent between about 0.5-100 μg/cm²-hr for a period of at least about 2 days. In another embodiment, the delivery system provides an in vitro skin flux of an active agent between about 0.5-80 μg/cm²-hr, 1-80 μg/cm²-hr, 2-80 μg/cm²-hr, 2-50 μg/cm²-hr, 4-50 μg/cm²-hr, 4-30 μg/cm²-hr, 0.5-15 μg/cm²-hr, 1-15 μg/cm²-hr or 4-15 μg/cm²-hr for a period of at least about 1 day, about 2 days, about 3 days, or about 4 days.

In embodiments, a release liner is at least partially in contact with at least one of the adhesive layers to protect the adhesive layer(s) prior to application. A release liner is typically a disposable layer that is removed prior to application of the device to the treatment site. In some embodiments, a release liner does not absorb components of the adhesive layer(s), including the active agent. In some embodiments, a release liner is impermeable to components of the adhesive layer(s) (including the active agent) and prevents release of components of the adhesive layer(s) through the release liner. In some embodiments, a release liner is formed of one or more of a film, non-woven fabric, woven fabric, laminate, and/or combinations thereof. In some embodiments, a release liner is a silicone-coated polymer film or paper. In some non-limiting embodiments, a release liner is a silicone-coated polyethylene terephthalate (PET) film, a fluorocarbon film, a fluorocarbon coated PET film, and/or combinations thereof.

Transdermal devices and systems (e.g., patches) may be prepared by any suitable methods as known in the art. In some general embodiments, transdermal devices are prepared by coating an appropriate amount of an adhesive polymer composition (with or without an active agent) onto a substrate such as a release liner or a backing layer. In some embodiments, the adhesive polymer composition is coated onto the release liner. In some embodiments, the adhesive polymer composition is coated onto the substrate or liner to a desired thickness. The thickness and/or size of the device and/or adhesive matrix may be determined by one skilled in the art based at least on considerations of wearability and/or required dose. It will be appreciated that the administration site for the device will affect the wearability considerations due to the available size of the administration site and the use of the administration site (e.g., need for flexibility to support movement). In some embodiments, the device and/or adhesive matrix has a thickness of between about 25-500 μm. The adhesive polymer composition and substrate are at least partially dried to remove any solvents. A release liner or backing layer is applied to the opposite side of the substrate. Where the substrate is not a release liner or backing layer, the substrate is replaced with the appropriate release liner or substrate. In embodiments that include multiple adhesive polymer layers, a first adhesive polymer composition is applied or coated onto the substrate, a tie layer material is applied to the formulation, and the second adhesive polymer composition is applied to the tie layer material. Adhesive polymer compositions and tie layers are laminated using any suitable methods known in the art. In some embodiments, adhesive layers are coated onto separate substrates or liners and then joined to form the transdermal delivery device. Where the delivery device includes a reservoir adhesive layer and a contact adhesive layer, adhesive polymer compositions may be coated onto the substrate or liner and laminated. It will be appreciated that any or all of the adhesive polymer composition layers may be dried before laminating the layers.

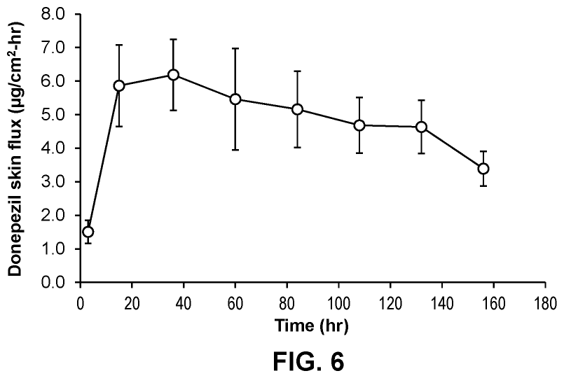

Transdermal delivery devices were prepared to demonstrate the delivery of an active agent formulated from an amine salt form of the active agent and an amphoteric inorganic base compound. The model amine salt form of the active agent was donepezil hydrochloride (Example 1) or memantine hydrochloride (Example 2) and the model amphoteric inorganic base compound was sodium bicarbonate. Other suitable examples of amphoteric inorganic base compounds include sodium carbonate, potassium carbonate, potassium bicarbonate, trisodium phosphate, disodium hydrogen phosphate, sodium oxylate, sodium succinate, sodium citrate, and sodium salicylate. As described in Example 1, a donepezil transdermal delivery system, for transdermal delivery of donepezil base, was prepared using donepezil hydrochloride and sodium bicarbonate in an adhesive matrix (polyacrylate) to form a drug reservoir. The drug reservoir was fabricated into a transdermal delivery system as depicted in FIG. 4, to have a drug-free contact adhesive layer for contact with the skin and a rate controlling membrane situated between the drug reservoir and the contact adhesive layer. A backing layer and a release liner completed the transdermal system. The donepezil transdermal system was evaluated in vitro by measuring release of donepezil from the system and across human skin, as detailed in Example 1. The donepezil skin flux rate is shown in FIG. 6. About 20 hours after application of the transdermal system to the skin, a steady-state flux rate of between about 4.8-6.4 μg/cm²-hr was achieved. The flux rate remained steady for about 5.5 days before decreasing. Accordingly, in one embodiment, a transdermal delivery system for delivery of a base form of an active agent is prepared from an amine salt form of the active agent and sodium bicarbonate, to provide a skin flux rate or permeation rate that is therapeutic for a period of at least about 3 days or 5 days or 7 days (or from 3-7 days). In one embodiment, the steady state in vitro skin flux rate remains within 15%, 20%, 25%, or 30% for a period of at least about 3 days or 5 days or 7 days (or from 3-7 days). That is, the in vitro skin flux measured at time point y varies from the in vitro skin flux measured at an earlier adjacent time point x, where x and y are each time points within a 3 day, 5 day, or 7 day measurement period, by less than about 20%, 25% or 30%.

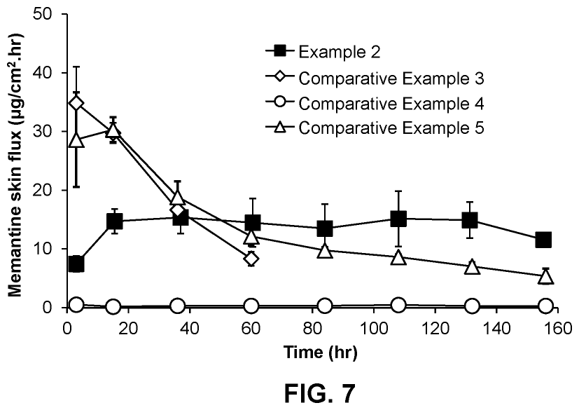

A memantine transdermal system was prepared as described in Example 2. The memantine transdermal system was evaluated in vitro by measuring release of memantine from the system and across human skin and the results are shown in FIG. 7 (squares). About 18 hours after application of the transdermal system to the skin, a steady-state flux rate of between about 12-15 μg/cm²-hr was achieved. The flux rate remained steady for about 6.5 days before decreasing. Accordingly, in one embodiment, a transdermal delivery system for delivery of a base form of an active agent is prepared from an amine salt form of the active agent and sodium bicarbonate, to provide a skin flux rate or permeation rate that is therapeutic for a period of at least about 3 days or 5 days or 7 days (or from 3-7 days). In one embodiment, the steady state in vitro skin flux rate remains within 15%, 20%, 25%, or 30% for a period of at least about 3 days or 5 days or 7 days (or from 3-7 days). That is, the in vitro skin flux measured at time point y varies from an in vitro skin flux measured at an earlier adjacent time point x, where x and y are each time points within a 3 day, 5 day, or 7 day measurement period, by less than 15%, 20%, 25% or 30%.

Comparative examples were also conducted to illustrate the inventive composition, system and methods described herein Data for comparative Examples 3-5 is included in FIG. 7 to illustrate that adhesive compositions (transdermal systems) prepared with the free base form of the drug (Comparative Example 3), with the amine salt form of drug but without sodium bicarbonate (Comparative Example 4) or a salt form of an amine drug and an amphoteric inorganic base compound, but where the pKa of the amphoteric inorganic base compound is not lower than that of the amine salt form of the active agent but is higher (Comparative Example 5). In these comparative examples, the in vitro skin flux of the drug is insufficient for therapy (FIG. 7, diamonds, circles, triangles, corresponding to Comparative Examples 3, 4, and 5, respectively).

### III. Methods of Treatment

In other aspects, methods of treating a disease, condition, and/or disorder by transdermal administration of at least one active agent by the transdermal compositions, devices, and/or systems described herein.

In some embodiments, compositions as described herein comprising donepezil (ARICEPT®) as an active agent are used for treating Alzheimer's disease, e.g., through administration of donepezil by a transdermal patch. Contemplated daily doses of donepezil are 5 mg, 10 mg, and 23 mg, with a range of daily dose from about 1-30 mg and about 2.5-25 mg and about 5-23 mg.

In some embodiments, compositions as described herein comprising memantine (NAMENDA®) as an active agent are used for treating Alzheimer's disease, obsessive compulsive disorder, anxiety disorder, ADHD, and opioid dependence, e.g., through administration of memantine by a transdermal patch. Contemplated daily doses of memantine are 2 mg, 5 mg, 7 mg, 10 mg, 14 mg, 21 mg, and 28 mg, with a range of daily dose from about 1-30 mg and about 1-28 mg.

In some embodiments, compositions as described herein comprising rotigotine (NEUPRO®) as an active agent are used for treating Parkinson's disease and restless leg syndrome, e.g., through administration of rotigotine by a transdermal patch. Contemplated daily doses of rotigotine are 1 mg, 2 mg, 3 mg, 4 mg, 6 mg, and 8 mg, with a range of daily dose from about 0.5-10 mg and about 1-8 mg.

In some embodiments, compositions as described herein comprising ropinirole (REQUIP®, REPREVE®, RONIROL®, ADARTREL®) as an active agent are used for treating Parkinson's disease and restless leg syndrome, e.g., through administration of ropinirole by a transdermal patch. Contemplated daily doses of ropinirole are of 0.25 mg, 0.5 mg, 1 mg, 2 mg, 3 mg, 4 mg, 5 mg, 6 mg, 8 mg, and 12 mg, with a range of daily dose from about 0.1-15 mg and about 0.25-15 mg.

In some embodiments, compositions as described herein comprising rivastigmine (EXELON®) as an active agent are used for treating Alzheimer's disease and/or Parkinson's disease dementia, e.g., through administration of rivastigmine by a transdermal patch. Contemplated daily doses of rivastigmine are 1.5 mg, 2.0 mg, 3.0 mg, 4.5 mg, 4.6 mg, 6.0 mg, 9.0 mg, 9.5 mg, and 13.3 mg, with a range of daily dose from about 0.5-18 mg, about 1-15 mg and about 1.5-13.3 mg.

In some embodiments, compositions as described herein comprising tamsulosin (FLOMAX®) as an active agent are used for treating benign prostatic hyperplasia, acute urinary retention, passage of kidney stones, e.g., through administration of tamsulosin by a transdermal patch. Contemplated daily doses of tamsulosin are 0.4 mg and 0.5 mg, with a range of daily dose from about 0.1-1.0 mg, about 0.2-0.8 mg and about 0.4-0.5 mg.

In some embodiments, compositions as described herein comprising methylphenidate (RITALIN®) as an active agent are used for treating attention deficit hyperactivity disorder (ADHD), narcolepsy, and/or depression, e.g., through administration of methylphenidate by a transdermal patch. Contemplated daily doses of methylphenidate are 2.5 mg, 5 mg, 10 mg, 15 mg, 18 mg, 20 mg, 27 mg, 30 mg, 36 mg, 40 mg, 50 mg, 54 mg, and 60 mg, with a range of daily dose from about 1-75 mg, about 2-65 mg, and about 2.5-60 mg.

In some embodiments, compositions as described herein comprise buprenorphine (CIZDOL®, SUBUTEX®, TEMGESIC®, BUPRENEX®, NORSPAN®, BUTRANS®) as an active agent are used for treating opioid addiction, moderate acute pain in non-opioid-tolerant individuals, and moderate chronic pain, e.g., through administration of buprenorphine by a transdermal patch. Contemplated doses of buprenorphine are 5 μg/hr, 7.5 μg/hr, 10 μg/hr, 15 μg/hr, 20 μg/hr, 0.075 mg, 0.15 mg, 0.3 mg, 0.36 mg, 0.45 mg, 0.5 mg, 0.6 mg, 0.7 mg, 0.71 mg, 0.75 mg, 0.9 mg, 1 mg, 1.4 mg, 2 mg, 2.1 mg, 2.9 mg, 3 mg, 4 mg, 4.2 mg, 5.7 mg, 6.3 mg, 8 mg, 8.6 mg, 11.4 mg, 12 mg, 80 mg, with a range of daily dose from about 0.01-100 mg, about 0.1-100 mg, about 0.075-80 mg, and about 0.075-15 mg.

In other embodiments, compositions as described herein comprise fentanyl (DURAGESIC®) as an active agent and are used for the relief of chronic pain and for managing severe chronic pain and for relief of moderate or severe chronic pain. Contemplated doses of fentanyl are 12.5 μg/hr, 25 μg/hr, 37.5 μg/hr, 50 μg/hr, 62.5 μg/hr, 75 μg/hr, 87.5 μg/hr, and daily doses of 0.3 mg, 0.6 mg, 0.9 mg, 1.2 mg, 1.5 mg, 1.8 mg, and 2.1 mg, with a range of daily dose from about 0.1-5 mg, about 0.1-2.5 mg, about 0.2-2.5, and about 0.3-2.1 mg.

In other embodiments, compositions as described herein comprise oxybutynin and are for use in reducing muscle spasms of the bladder and urinary tract, for treating symptoms of overactive bladder, including frequent or urgent urination, incontinence and increased night-time urination. Contemplated daily doses of oxybutynin are 3.9 mg, 5 mg, 10 mg, 15 mg, and 20 mg and 30 mg, with a dosage range of about 1-35 mg, about 2-35 mg, about 3-30 mg, and about 3.9-30 mg.

In other embodiments, compositions as described herein comprise fingolimod and are for use in treating multiple sclerosis or relapsing-remitting multiple sclerosis.

Transdermal compositions, devices, and/or systems described herein may be designed for long term use and/or continuous administration of at least one active agent. It will be appreciated that the total dose of the active agent per transdermal device will be determined by the nature of the active agent(s), the size of the device, and/or the loading of the active agent within the adhesive matrix. In some embodiments, the application period for the transdermal device is between about 1-10 days, 1-7 days, 1-5 days, 1-2 days, 1-3 days, 1-4 days, 3-10 days, 3-7 days, 3-5 days, 5-10 days, and 5-7 days, inclusive. In some embodiments, the active agent is released from the adhesive matrix as a continuous and/or sustained release over the application period.

### IV. Examples

The following examples are illustrative in nature and are in no way intended to be limiting.

Unless otherwise specified, the following materials were used in the examples described below: the backing layer was SCOTCHPAK™ 9723; the release liner was a silicone-coated polyester (PET) film; the nonwoven tie layer was REEMAY® 2250; the rate controlling membrane was CELGARD® 2400 microporous polypropylene; and the contact adhesive layer was acrylate, polyisobutylene (PIB), and/or silicone adhesive.

### Example 1

Donepezil Salt Transdermal Formulation with Sodium Bicarbonate

### Preparation of Drug-in-Adhesive

An amount of 1.20 g of SPAN® 20 was dissolved in 6.00 g of triethyl citrate, and the solution was mixed with 1.80 g of lauryl lactate and 89.69 g of ethyl acetate. To the solution was added 6.00 g of glycerine and was mixed well. To the mixture, 9.00 g of donepezil hydrochloride and 1.82 g of sodium bicarbonate were dispersed. After addition of 12.00 g of KOLLIDON® CL-M to the drug dispersed solution, the mixture was homogenized by a Silverson mixer homogenizer. To the homogenized drug dispersion, 43.93 g of DURO-TAK® 387-2287 (solid content 50.5%) was added and mixed well. The wet adhesive formulation was coated on a release liner and dried using a Werner Mathis lab coater to get a dry coat weight of 12 mg/cm².

### Preparation of Contact Adhesive

An amount of 0.60 g of sorbitan monolaurate (SPAN® 20) was dissolved in 3.00 g of triethyl citrate, and mixed with 0.9 g of lauryl lactate, 25.45 g of ethyl acetate, and 1.34 grams of isopropyl alcohol. After addition of 6.00 g of crosslinked polyvinylpyrrolidone (KOLLIDON® CL-M), the mixture was homogenized by a Silverson homogenizer. To the homogenized mixture, an amount of 38.61 g of DURO-TAK® 387-2287 (solid content 50.5%) was added and mixed well. The wet adhesive formulation was coated on a release liner and dried to give a dry coat weight of 5 mg/cm² using a Werner Mathis lab coater.

### Lamination and Die-cut

A microporous polypropylene membrane (CELGARD® 2400) was laminated between the drug-in-adhesive and the contact adhesive layers. The release liner on the drug reservoir side was replaced and laminated with a backing film, 3M SCOTCHPAK® 1012. The final five layer laminate was die-cut into patches. FIG. 4 depicts the design of the patch described in Example 1.

### Evaluation of In Vitro Skin Flux

Dermatomed human cadaver skin was obtained from a skin bank and frozen until ready for use. The skin was placed in water at 60° C. for 1-2 minutes after thawing and the epidermis carefully separated from dermis. The epidermis was either used immediately or wrapped and frozen for later use.

In vitro skin flux studies were performed using a Franz type diffusion cell with an active diffusion area of 0.64 cm². The epidermis was mounted between the donor and receptor compartments of the diffusion cell. The transdermal delivery system was placed over the skin and the two compartments were clamped tight together.

The receptor compartment was filled with 0.01 M phosphate buffer, pH 6.5, containing 0.01% gentamicin. The solution in the receptor compartment was continually stirred using a magnetic stirring bar in the receptor compartment. The temperature was maintained at 32±0.5° C. Samples were drawn from the receptor solution at periodic intervals and the receptor solution was replaced with fresh phosphate buffers solution. Drug content in the samples was analyzed using HPLC for donepezil. The flux profile results are shown in FIG. 6. The flux in this example is relatively high and remains relatively constant over 7 days.

### Example 2

Memantine Salt Transdermal Formulation with Sodium Bicarbonate

### Preparation of Drug-in-Adhesive

An amount of 2.0 g of glycerine and 2.0 g of octyl dodecanol were mixed with a mixture of 29.35 g of ethyl acetate and 1.86 g of isopropyl alcohol. In the solution, 5.0 g of memantine hydrochloride and 1.95 g of sodium bicarbonate were dispersed by stirring. To the dispersion, 3.0 g of crosslinked polyvinylpyrrolidone (KOLLIDON® CL-M) was added and homogenized using a Silverson mixer homogenizer. To the homogenized drug dispersion, 11.99 g of acrylate copolymer (DURO-TAK® 387-2287, solid content 50.5%) was added and mixed well. The wet adhesive formulation was coated on a release liner and dried using a Werner Mathis coater to get a dry coat weight of 15 mg/cm².

### Preparation of Contact Adhesive

An amount of 2.0 g of octyl dodecanol was mixed with 20.67 g of n-heptane. After addition of 4.00 g of crosslinked polyvinylpyrrolidone (KOLLIDON® CL-M) to the solution, the mixture was homogenized using a Silverson mixer homogenizer. To the homogenized mixture, an amount of 23.33 g of polyisobutylene adhesive solution (solid content 60%) was added and mixed well. The wet adhesive formulation was coated on a release liner and dried to give a dry coat weight of 5 mg/cm².

### Lamination and Die-Cut

A microporous polypropylene membrane (CELGARD® 2400) was laminated between the drug-in-adhesive layer and the contact adhesive layer. The release liner on the drug-in-adhesive side was replaced and laminated with a backing, 3M SCOTCHPAK® 1012. The final five layer laminate was die-cut into patches. FIG. 4 depicts the design of the patch.

### Evaluation of In Vitro Skin Flux

The in vitro skin flux of memantine from the transdermal system was measured as described in Example 1, where memantine content in the samples drawn from receptor solution were analyzed for memantine using liquid chromatography mass spectrometry (LCMS). The flux profile results are shown in FIG. 7 (squares). The flux in this example is relatively high and remains relatively constant over 7 days.

### Comparative Example 3

### Memantine Free Base Transdermal Formulation

### Preparation of Drug-in-Adhesive

An amount of 3.20 g of memantine free base was mixed in a mixture of 5.48 g of ethyl acetate and 8.23 g of n-heptane. To the mixture, 4.00 grams of crosslinked polyvinylpyrrolidone (KOLLIDON® CL-M) was dispersed and homogenized by a Silverson mixer homogenizer. To the homogenized dispersion, 29.09 g of polyisobutylene (PIB) adhesive solution (solid content 44%) was added and well mixed. The wet adhesive formulation was coated on a release liner and dried using a Werner Mathis lab coater to get a dry coat weight of 9 mg/cm².

### Lamination and Die-Cut

A nonwoven polyethylene terephthalate fabric, REEMAY® 2250, was inserted between two drug-in-adhesive layers. One release liner was replaced with a backing, SCOTCHPAK® 1012 film. The final five layer laminate was die-cut into patches. FIG. 4 depicts the design of the patch described in the comparative example.

### Evaluation of In Vitro Skin Flux

The in vitro skin flux of memantine from the transdermal system was measured as described in Example 1, where memantine content in the samples drawn from receptor solution were analyzed for memantine using LCMS. The flux profile results are shown in FIG. 7 (diamonds). Memantine free base diffuses through the matrix and skin very quickly and is depleted in a relatively short time. The flux is difficult to control and to maintain at a constant rate for multiple days.

### Comparative Example 4

Memantine Salt Transdermal Formulation without Sodium Bicarbonate

### Preparation of Drug-in-Adhesive

An amount of 7.5 g of memantine HCl and 7.5 g of crosslinked polyvinylpyrrolidone (KOLLIDON® CL-M) were homogenized in 46.46 g of ethyl acetate with a Silverson mixer homogenizer. The homogenized dispersion was mixed well with 81.4 g of acrylate copolymer (DURO-TAK® 87-900A acrylate adhesive solution (43% solid)). The solution was coated and dried using a Werner Mathis Lab coater to get a coat weight of 9 mg/cm². A porous polyethylene film, DELNET® X540NAT was inserted between two drug-in-adhesive layers. One of adhesive release liners was replaced with a backing, 3M SCOTCHPAK® 1012.

### Preparation of Contact Adhesive

An amount of 7.5 g of crosslinked polyvinylpyrrolidone (KOLLIDON® CL-M) was homogenized in 36.52 g of ethyl acetate with a Silverson mixer homogenizer. The homogenized dispersion was mixed well with 98.84 g of DURO-TAK® 87-900A (43% solid content) acrylate adhesive solution.

### Lamination and Die-Cut

The release liner of drug-in-adhesive layer was removed and the contact adhesive layer was laminated directly to the drug-in-adhesive layer. The six layer laminate was die-cut into patches. FIG. 3 depicts the design of the patch described in the comparative example.

### Evaluation of In Vitro Skin Flux

The in vitro skin flux of memantine from the transdermal system was measured as described in Example 1, where memantine content in the samples drawn from receptor solution were analyzed for memantine using LCMS. The flux profile results are shown in FIG. 7 (circles). Skin flux of memantine salt in this example is negligible. The conjugated acid salt form of memantine is barely permeable through the in vitro human skin.

### Comparative Example 5

Memantine Salt Transdermal Formulation with Sodium Carbonate

### Preparation of Drug-in-Adhesive

An amount of 2.0 g of glycerine and 2.0 g of triethyl citrate were mixed with a mixture of 26.44 g of ethyl acetate and 1.70 g of isopropyl alcohol. In the solution, 5.0 g of memantine hydrochloride and 1.96 g of sodium carbonate were dispersed by stirring. To the dispersion, 3.0 g of crosslinked polyvinylpyrrolidone (KOLLIDON® CL-M) was added and homogenized using a Silverson mixer homogenizer. To the homogenized drug dispersion, 11.95 g of acrylate copolymer (DURO-TAK® 387-2287 (solid content 50.5%)) was added and well mixed. The wet adhesive formulation was coated on a release liner and dried using a Werner Mathis coater to get a dry coat weight of 15 mg/cm².

### Preparation of Contact Adhesive

An amount of 2.0 g of triethyl citrate was mixed with a mixture of 14.78 g of ethyl acetate and 1.50 g of isopropyl alcohol. After addition of 4.00 g of crosslinked polyvinylpyrrolidone (KOLLIDON® CL-M) to the solution the mixture was homogenized by a Silverson mixer homogenizer. To the homogenized mixture an amount of 27.72 g of acrylate copolymer (DURO-TAK® 387-2287 acrylic adhesive solution (solid content 50.5%)) was added and mixed well. The wet adhesive formulation was coated on a release liner and dried to give a dry coat weight of 5 mg/cm².

### Lamination and Die-Cut

A microporous polypropylene membrane (CELGARD® 2400) was laminated between the drug-in-adhesive layer and the contact adhesive layer. The release liner on the drug-in-adhesive side was replaced and laminated with a backing, 3M SCOTCHPAK® 1012. The final five layer laminate was die-cut into patches. FIG. 4 depicts the design of the patch described in this comparative example.

### Evaluation of In Vitro Skin Flux

The in vitro skin flux of memantine from the transdermal system was measured as described in Example 1, where memantine content in the samples drawn from receptor solution were analyzed for memantine using LCMS. The flux profile results are shown in FIG. 7 (triangles). The flux profile is similar to that of the free base formulation of Example 3. Sodium carbonate has pKa of 10.3, which is much higher than that of sodium bicarbonate, and is similar to that of conjugated acid memantine salt (pKa 10.27). Therefore, the pKa ratio between sodium carbonate and memantine HCl is close to one, at which almost an equal fraction of the memantine free base is produced relative to the memantine salt. As such, this patch behaves like a free base formulation. If the free base is a solid form, it will be recrystallized as this reaction proceeds, and the skin flux will rapidly drop to zero as crystals form in the drug-in-adhesive.

### EQUIVALENTS

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

All patents, patent applications, patent publications, and other publications mentioned herein are hereby incorporated by reference in their entirety. Where a patent, application, or publication contains express definitions, those definitions should be understood to apply to the incorporated patent, application or publication in which they are found and not to the present application unless otherwise indicated.

It is claimed:

1. A transdermal delivery system, comprising:
a drug reservoir comprising an amine salt form of donepezil and an amphoteric inorganic base compound selected from sodium bicarbonate and potassium bicarbonate, wherein the pKa of the amphoteric inorganic base compound is lower than that of the amine salt form of the active agent.

2. The transdermal delivery system of claim 1, wherein the amphoteric inorganic base compound is sodium bicarbonate.

3. The transdermal delivery system of claim 1, wherein the amine salt form of donezpezil is present in the drug reservoir in an amount between about 5-35% w/w.

4. The transdermal delivery system of claim 1, wherein the sodium bicarbonate is present in the drug reservoir in an amount between about 0.5-35% w/w.

5. The transdermal delivery system of claim 1, wherein the amine salt form of donezpezil is donepezil HCl, and wherein the drug reservoir comprises a molar amount of donepezil HCl and a less than equimolar amount of sodium bicarbonate.

6. The transdermal delivery system of claim 1, wherein the drug reservoir further comprises a salt form solubilizer selected from the group consisting of water, alcohols, glycerol, propylene glycol, ethylene glycol, dimethyl sulfoxide, and N-methylpyrrolidone.

7. The transdermal delivery system of claim 6, wherein the salt form solubilizer is present in the drug reservoir in an amount of up to 15% w/w.

8. The transdermal delivery system of claim 1, wherein the drug reservoir further comprises a neutral form solubilizer selected from the group consisting of a fatty acid ester, a dicarboxylic acid ester, a glycerol ester, a lactate, and a fatty alcohol.

9. The transdermal delivery system of claim 1, wherein the drug reservoir further comprises a neutral form solubilizer selected from the group consisting of sorbitan monolaurate, lauryl lactate, and triethyl citrate.

10. The transdermal delivery system of claim 9, wherein the neutral form solubilizer is present in the drug reservoir in an amount of up to 20% w/w.

11. The transdermal delivery system of claim 1, wherein the drug reservoir further comprises an additive selected from the group consisting of crospovidone and colloidal silicone dioxide.

12. The transdermal delivery system of claim 11, wherein the additive is present in the drug reservoir in an amount of up to 25% w/w.

13. The composition of claim 1, wherein the drug reservoir further comprises an acrylate polymer or copolymer adhesive agent.

14. The transdermal delivery system of claim 13, wherein the adhesive agent comprises up to 65% w/w of the drug reservoir.

15. The transdermal delivery system of claim 1, further comprising a backing layer.

16. The transdermal delivery system of claim 15, wherein the backing layer is an occlusive polymer film.

17. The transdermal delivery system of claim 15, further comprising a contact adhesive layer comprised of an acrylate polymer or copolymer.

18. The transdermal delivery system of claim 17, further comprising a nonwoven tie layer between the drug reservoir and the contact adhesive layer.

19. The transdermal delivery system of claim 17, further comprising a rate-controlling membrane between the drug reservoir and the contact adhesive layer.

20. A method for treating Alzheimer's disease, comprising: providing a transdermal delivery system according to claim 1 to a patient in need thereof.

21. The method of claim 20, further comprising administering or instructing to administer to the skin of the patient the composition.

22. The method of claim 21, wherein said administering achieves a therapeutically effective blood concentration of donepezil.
